(19) United States
(12) Patent Application Publication
(10) Pub. No.: US 2012/0264824 A1
(43) Pub. Date: Oct. 18, 2012

(54) COMPOSITIONS AND METHODS FOR TREATING NON-ALCOHOLIC STEATOHEPATITIS

(75) Inventors: Kiyoshi MIZUGUCHI, Saitama (JP); Tsuyoshi Harada, Tokyo (JP)

(73) Assignee: MOCHIDA PHARMACEUTICAL CO., LTD., Tokyo (JP)

(21) Appl. No.: 13/088,072

(22) Filed: Apr. 15, 2011

(51) Int. Cl. A61K 31/232; A61P 1/16

(52) U.S. Cl. 514/549

(57) ABSTRACT

Methods and compositions are disclosed comprising ethyl eicosapentanoate for the treatment of non-alcoholic steatohepatitis (NASH).

### BACKGROUND OF THE INVENTION

1. Field of the Invention

The present invention relates to methods and compositions comprising ethyl eicosapentanoate for the treatment of non-alcoholic steatohepatitis (NASH).

2. Background

It is known that heavy alcohol use can lead to liver complications, including alcoholic hepatitis which is often characterized by fatty liver and inflammation. Alcoholic hepatitis can ultimately lead to cirrhosis of the liver (scarring) and hardening of the liver tissue.

Individuals that do not consume excessive amounts of alcohol can also be found to have liver disease complications. Non-alcoholic fatty liver disease (NAFLD) is understood to encompass a variety of liver diseases, including steatosis (simple fatty liver), non-alcoholic steatohepatitis (NASH) and advanced scarring of the liver (cirrhosis). NASH has traditionally been diagnosed by means of a liver biopsy to characterize the liver histology, particularly with respect to the characteristics of inflammation, fibrosis and steatosis (fat accumulation). NASH then generally prefers to clinical findings based upon the liver biopsy of a patient with steatohepatitis, combined with the absence of significant alcohol consumption (Neuschwander-Tetri, B. A. and S. H. Caldwell (2003) Hepatology 37(5): 1202-1209). In NASH, fat accumulation is seen in varying degrees of inflammation (hepatitis) and scarring (fibrosis). Patients having NASH are also often characterized by abnormal levels of liver enzymes, such as aspartate aminotransferase (AST) and alanine aminotransferase (ALT). However, a clinical diagnosis of NASH still depends upon a liver biopsy to assess the histologic characteristics of the patient's liver, such that histological examination of liver biopsy tissue is often characterized as the “gold-standard” technique for the assessment of liver fibrosis (Neuschwander-Tetri, ibid).

### SUMMARY OF THE INVENTION

The present invention relates to methods and compositions comprising ethyl eicosapentanoate for the treatment or alleviation of non-alcoholic steatohepatitis (NASH), and alleviation of the symptoms associated with NASH.

In one embodiment of the invention a patient in need of treatment for NASH is administered an effective amount of ethyl eicosapentanoate, wherein the patient has a baseline level indicative of NASH of at least one criteria selected from the group consisting of NAS score, steatosis score, lobular inflammation score, ballooning score and fibrosis stage.

In another embodiment of the invention the method for treating NASH comprises identifying a subject having NASH; determining the baseline level in the subject of at least one criteria selected from the group consisting of NAS score, steatosis score, lobular inflammation score, ballooning score and fibrosis stage; and administering to the subject an effective amount of ethyl eicosapentanoate.

In another embodiment of the invention the method for treating NASH comprises identifying a subject/patient having NASH; determining the baseline level in the subject of at least one criteria selected from the group consisting of NAS score, steatosis score, lobular inflammation score, ballooning score and fibrosis stage; administering to the subject an effective amount of ethyl eicosapentanoate; and improving the NAS score (i) to a composite score of less than or equal to 3 or (ii) by 2 across at least two of the NAS components, combined with no worsening of the fibrosis stage score.

In another embodiment of the invention the method for treating NASH comprises identifying a subject having NASH characterized by baseline levels of ALT of between 5 to 300 and at least one criteria selected from the group consisting of NAS score of ≧4, a steatosis score of ≧1, a lobular inflammation score of ≧1 and either (i) a fibrosis stage of at least 1a or (ii) ballooning; administering to the subject an effective amount of ethyl eicosapentanoate; and improving the NAS score in the subject (i) to a composite score of ≦3 or (ii) by ≧2 across at least two of the NAS components, together with no worsening of the fibrosis stage score.

In a further embodiment of the invention, the method for treating NASH comprises administering to a subject an effective amount of ethyl eicosapentanoate, wherein the subject has NASH characterized by baseline levels of ALT of between 5 to 300 and at least one criteria selected from the group consisting of a NAS score of ≧4, a steatosis score of ≧1, lobular inflammation score of ≧1 and either (i) a fibrosis stage of at least 1a or (ii) ballooning; and improving the NAS score in the subject (i) to a composite score of ≦3 or (ii) by ≧2 across at least two of the NAS components, together with no worsening of the fibrosis stage score.

In another embodiment of the invention, the method for reducing steatosis, liver lobular inflammation, ballooning and/or liver fibrosis in a subject in need thereof, comprising administering to a subject an effective amount of ethyl eicosapentanoate (EPA-E); improving at least one condition selected from the group consisting of the steatosis, lobular inflammation, ballooning and liver fibrosis condition of said subject, and no worsening of said fibrosis stage score; and said subject exhibits the following changes in said at least one marker as compared to a baseline pretreatment level of at least 1% reduction for ALT, AST, Triglycerides (TG), TG/HDL-C ratio, Free fatty acid, Arachidonic acid (AA), monounsaturated fatty acid (MUFA), Palmitoleic acid, Oleic acid, Oleic Acid/Stearic acid ratio, Palmitoleic acid/Palmitic acid ratio, Stearic acid/Palmitic acid ratio, γ-linoleic acid/Linoleic acid ratio, Adrenic acid/AA ratio, Ferritin, Thioredoxin, TNF α, sTNF-R1, sTNF-R2, Hs-CRP, CTGF, sCD40, Leptin, complement factor D, CK18 fragment, serum HMGB1, Fas, Hyaluronic acid, Type IV collagen (7s domain), procollagen III peptide or PAl-1; at least 5% increase for EPA or EPA/AA ratio; at least 1% increase for DPA, AA/Homo-γ-linoleic acid ratio or Serum adiponectin; no worsening of ALP, bilirubin, GGT, Albumin, HDL-C, LDL-C, Total Cholesterol (TC), non-HDL-C, HOMA-IR, HbAlc, Fasting plasma glucose, postprandial plasma glucose, OGTT, platelet count or BMI.

In another embodiment of the invention, the method for treating NASH comprises administering to a subject an effective amount of ethyl eicosapentanoate, wherein the subject is possible or definite NASH, and the subject is determined prior to treatment a baseline level in blood or physical condition of at least one member selected from the group consisting of ALT, AST, AST/ALT ratio, ALP, bilirubin, GGT, Albumin, HDL-C, LDL-C, TG, TC, TG/HDL-C ratio, non-HDL-C, Free fatty acid, AA, EPA, DPA, DHA, EPA/AA ratio, DPA/AA ratio, DHA/AA ratio, DHA/DPA ratio, MUFA, Palmitoleic acid, Oleic acid, Oleic acid/Stearic acid ratio, Palmitoleic acid/Palmitic acid ratio, Stearic acid/Palmitic acid ratio, y-linoleic acid/Linoleic acid ratio, AA/Homo-γ-linoleic acid ratio, Adrenic acid/AA ratio, Ferritin, Thioredoxin, TNF α, sTNF-R1, sTNF-R2, Hs-CRP, CTGF, sCD40, HOMA-IR, HbAlc, Glucose, Fasting plasma glucose, postprandial plasma glucose, OGTT, Leptin, Serum adiponectin, complement factor D, CK18 fragment, serum HMGB1, Fas, Hyaluronic acid, Type IV collagen (7s domain), procollagen III peptide, PAl-1, platelet count or BMI.

In another embodiment of the invention, the method for treating NASH comprises administering to a subject an effective amount of ethyl eicosapentanoate, wherein the subject is possible or definite NASH, and exhibits the following changes in said at least one marker as compared to a baseline pre-treatment level of at least 1% reduction for ALT, AST, TG, TG/HDL-C ratio, Free Fatty acid, AA, MUFA, Palmitoleic acid, Oleic acid, Oleic acid/Stearic acid ratio, Palmitoleic acid/Palmitic acid ratio, Stearic acid/Palmitic acid ratio, y-linoleic acid/Linoleic acid ratio, Adrenic acid/AA ratio, Ferritin, Thioredoxin, TNF α, sTNF-R1, sTNF-R2, Hs-CRP, CTGF, sCD40, Leptin, complement factor D, CK18 fragment, serum HMGB1, Fas, Hyaluronic acid, Type IV collagen (7s domain), procollagen III peptide or PAI-1; at least 5% increase for EPA or EPA/AA ratio; at least 1% increase for DPA, AA/Homo-γ-linoleic acid ratio or Serum adiponectin; no worsening of ALP, bilirubin, GGT, Albumin, HDL-C, LDL-C, TC, non-HDL-C, HOMA-IR, HbAlc, Glucose, Fasting plasma glucose, postprandial plasma glucose, OGTT, platelet count or BMI.

In another embodiment of the invention, the method for treating NASH comprises administering to a subject an effective amount of ethyl eicosapentanoate, wherein the subject is taking anti-diabetic drugs.

In another embodiment of the invention, the method for treating NASH comprises administering to a subject an effective amount of ethyl eicosapentanoate, wherein the subject is not taking anti-diabetic drugs.

In another embodiment of the invention, the method for treating NASH comprises administering to a subject an effective amount of ethyl eicosapentanoate, wherein the subject is not diabetic.

In another embodiment of the invention, the method for reducing at least one marker as compared to a baseline pre-treatment level of Hs-CRP, CTGF, sCD40, Leptin, complement factor D, serum HMGB1, Fas or procollagen III peptide in a subject, comprising administering to a subject an effective amount of ethyl eicosapentanoate (EPA-E), wherein the subject has NASH.

In another embodiment of the invention, the method of determining efficacy of NASH treatment by (i) administering to a subject an effective amount of EPA-E, (ii) measuring at least one marker in Table 1 during the treatment, (III) comparing the measured levels of markers to established levels in advance, and optionally (iv) determining whether the treatment is efficacious.

### DETAILED DESCRIPTION OF THE INVENTION

The methods and compositions of the present invention are useful for the treatment of NASH by administration of an effective amount of ethyl eicosapentanoate.

Eicosapentaenoic acid (EPA) is a known omega-3 polyunsaturated, long-chain fatty acid. Omega-3 fatty acids are known as components of oils, such as fish oil, and a variety of commercial products are promoted as containing omega-3 fatty acids, or their esters, derivatives, conjugates and the like. Eicosapentaenoic acid (EPA) is also per se known in its ethyl ester form, ethyl eicosapentanoate (EPA-E). According to the present invention, EPA-E can be administered in a composition. EPA-E content in the total fatty acid of the compositions of the present invention are not particularly limited as long as the composition contains EPA-E as its effective component and intended effects of the present invention are attained, high purity EPA-E is preferably used; for example, the composition having a proportion of the EPA-E of preferably 40% by weight or more, more preferably 90% by weight or more, and still more preferably 96.5% by weight or more in total of the fatty acids and their derivatives. EPA-E can be administered to patients in a highly purified form, including the product known as Epadel® (Mochida Pharmaceutical Co., Ltd., Tokyo Japan). The compositions of EPA-E are administered according to the invention to a subject or patient to provide the patient with a dosage of about 0.3-10 g per day of EPA-E, alternatively 0.6-6 g per day, alternatively 0.9-3.6 g per day or specifically about 1800 mg per day or about 2700 mg per day of EPA-E.

The composition to be administered can contain other fatty acids, especially any omega-3 unsaturated fatty acid, especially DHA-E. The ratio of EPA-E/DHA-E in the composition, the content of EPA-E and DHA-E in the total fatty acids and administration amount of EPA-E and DHA-E are not limited but the ratio is preferably 0.8 or more, more preferably 1.0 or more, still more preferably 1.2 or more. The composition is preferably highly purified; for example, the proportion of EPA-E+DHA-E in the fatty acids and their derivatives is preferably 40% by weight or more, more preferably 80% by weight or more, and still more preferably 90% or more. The daily amount in terms of EPA-E+DHA-E is typically 0.3 to 10.0 g/day, preferably 0.5 to 6.0 g/day, and still more preferably 1.0 to 4.0 g/day. The low content of other long chain saturated fatty acids is preferred, and among the long chain unsaturated fatty acids, the content of omega-6 fatty acids, and in particular, the content of arachidonic acid is preferably as low as less than 2% by weight, and more preferably less than 1% by weight. For example, soft capsule (Lovaza™) containing about 46% by weight of EPA-E and about 38% by weight of DHA-E is commercially available in the U.S. and other countries as a therapeutic agent for hyerptriglyceridemia.

Patients treated for NASH can be administered EPA-E according to the invention for 3, 6 or 9 months, or for 1 year or more and can be administered EPA-E in one, two or three dosage per day, or other multiple doses per day including 1 to about 10, 1 to 8, 1 to 6, 1 to 4 or 1 to 2 dosage units per day as appropriate for patient therapy. The term “dose unit” and “dosage unit” herein refer to a portion of a pharmaceutical composition that contains an amount of EPA-E for a single administration to a subject.

Compositions comprising EPA-E useful for the invention include commercially available compositions of EPA-E, such as Epadel® noted above. Compositions comprising EPA-E may be administered in tablet, capsule, powder or any other solid oral dosage form, as a liquid, as a soft gel capsule or other capsule form, or other appropriate and convenient dosage forms for administration to a patient in need thereof. Compositions can also include pharmaceutically acceptable excipients known to those of ordinary skill in the art including surfactants, oils, co-solvents or combinations of such excipients, together with stabilizers, emulsifiers, preservatives, solubilizers and/or other non-active pharmaceutical ingredients known to those of skill in the art relative to the preparation of pharmaceutical compositions.

1. Evaluation Criteria for Patients

As noted above, the “gold-standard” for a complete diagnosis of NASH involves a liver biopsy. Patients or subjects treated for NASH according to the present invention can also be evaluated for the following criteria, including evaluation prior to initiation of treatment in order to provide a baseline level or score for the criteria as well as evaluation after the dosing regimen to evaluate any improvement in the criteria.

a. NAS Score:

A non-alcoholic fatty liver disease activity score (NAS) is defined as the unweighted sum of the values for steatosis (ranging from 0-3), lobular inflammation (ranging from 0-3) and ballooning (ranging from 0-2), thereby providing a range of NAS score of from 0 to 8. (See Kleinen et al., Design and Validation of a Histological Scoring System for Nonalcoholic Fatty Liver Disease, Hepatology, Vol. 41, No. 6, 2005, pp. 1313-1321) Patients treated for NASH according to the present invention can show a NAS score prior to treatment of ≧4, with a minimum score of 1 each for steatosis and lobular inflammation plus either ballooning or at least 1 a sinusoidal fibrosis and a finding of possible or definite steatohepatitis. After dosing/treatment, such as for one year, patients can show a composite NAS score of ≦3, ≦2 or ≦1, together with no worsening in fibrosis. Alternatively, patients can show an improvement in NAS by a value of ≧2 across at least two of the NAS components, together with no worsening in fibrosis. Alternatively, patients can show an improvement in NAS score by ≧3, 4, 5, 6, 7 or 8.

b. Steatosis:

Steatosis is broadly understood to describe a process involving the abnormal retention of lipids within the liver, which accumulation inhibits the normal liver functions. Liver biopsy enables analysis and scoring of steatosis in a patient, with scores ranging from 0-3. Patients treated for NASH according to the present invention can have a steatosis score of 1, 2 or 3, such as between about 2 and about 3. After treatment, it is desired for patients to exhibit no worsening of steatosis, alternatively a reduction of at least 1 in the steatosis score, or a reduction of 2 or 3 in the steatosis score. Steatosis is traditionally graded with a score of 1 indicating the presence of fat droplets in less than 33% of hepatocytes, a score of 2 indicating fat droplets observed in 33-66% of hepatocytes, and a score of 3 indicating observation of fat droplets in greater than 66% of hepato sites. (See Kleinen et al., Design and Validation of a Histological Scoring System for Nonalcoholic Fatty Liver Disease, Hepatology, Vol. 41, No. 6, 2005, pp. 1313-1321)

c. Lobular Inflammation:

Lobular inflammation is also evaluated upon liver biopsy and scored with values of 0-3. (See Kleinen et al., Design and Validation of a Histological Scoring System for Nonalcoholic Fatty Liver Disease, Hepatology, Vol. 41, No. 6, 2005, pp. 1313-1321 Table 1) Patients to be treated for NASH can have lobular inflammation scores of 1, 2 or 3, alternatively ranging between 1 and 2 or 2 and 3. After treatment, patients can have a reduction in lobular inflammation score of at least 1, alternatively a reduction of 2 or 3 in lobular inflammation score, and at least no worsening of the lobular inflammation score.

d. Ballooning:

Ballooning of hepatocytes is generally scored with values of 0-2, (See Kleinen et al., Design and Validation of a Histological Scoring System for Nonalcoholic Fatty Liver Disease, Hepatology, Vol. 41, No. 6, 2005, pp. 1313-1321 Table 1), and patients treated for NASH according to the present invention can have ballooning scores of 0-2, including specific values of 1 or 2, and alternatively a score ranging from 1 to 2. After treatment, patients can show at least no worsening of the ballooning score, alternatively a reduction of at least one value lower in the ballooning score, and alternatively a reduction of two in the value of the ballooning score.

e. Fibrosis Stage

Fibrosis is also evaluated upon liver biopsy and scored with values of 0-4, the scores being defined as: 0 represents no fibrosis, 1 represents perisinusoidal or periportal fibrosis, 1a represents mild, zone 3, perisinusoidal fibrosis; 1b represents moderate zone 3, perisinusoidal fibrosis; 1c represents portal/periortal fibrosis; 2 represents perisinusoidal and portal/periportal fibrosis; 3 represents bridging fibrosis; and 4 represents cirrhosis. (See Kleinen et al., Design and Validation of a Histological Scoring System for Nonalcoholic Fatty Liver Disease, Hepatology, Vol. 41, No. 6, 2005, pp. 1313-1321) Patients treated according to the present invention can have a fibrosis stage score of 0-3, including 0, 1, 1a, 1 b, 1 c, 2 or 3, and can have a fibrosis stage score of at least 1a. After treatment, patients can have a fibrosis stage score that is at least no worse than the baseline score, and alternatively can have a reduction in the fibrosis stage score of at least one level, alternatively at least two or three levels.

2. Additional Criteria/Markers for Evaluation of Patients

As noted above, while liver biopsy is considered the “gold-standard” for clinical assessment of NASH, the condition can also be accompanied or associated with abnormal levels of liver enzymes and other biological blood components. Therefore, patients treated for NASH according to the present invention can also be evaluated for baseline scores of the following criteria before treatment, and evaluated after treatment for possible changes in those criteria. The evaluated criteria can comprise one or more of the following criteria set forth in Table 1.

TABLE 1

| | Pre-treatment baseline | | After dosing (effect) values | |
|---|---|---|---|---|
| Item (Typical | | Observable | | Observable |
| Normal | Typical | Ranges or | Typical | Ranges or |
| Values, Units) | Range(s) | Values | Range(s) | Values |
| ALT | 10-300 | Lower limit | at least 1% | 1 to about 95% |
| (6-41 U/L) | | range values of | lower | reduction |
| | | 10, 50, 100, 150, | | |
| | | or 200, upper | | |
| | | limit range | | |
| | | values of 100, | | |
| | | 150, 200, 250, | | |
| | | or 300, ranges | | |
| | | of 10-300, 10- | | |
| | | 200, 10-150, 10- | | |
| | | 100, 100-200, | | |
| | | 2000-3000 | | |
| AST | 10-250 | Lower limit | at least 1% | 1 to about 95% |
| (9-34 U/L) | | range values of | lower | reduction |
| | | 10, 50, 100, 150, | | |
| | | or 200, upper | | |
| | | limit range | | |
| | | values of 100, | | |
| | | 150, 200, 250, | | |
| | | or 300, ranges | | |
| | | of 10-300, 10- | | |
| | | 200, 10-150, 10- | | |
| | | 100, 100-200, | | |
| | | 200-300 | | |
| AST/ALT ratio | | upper limit | | |
| | | range values of | | |
| | | 0.5, 0.7, 0.8, 1, | | |
| | | 1.2, 2; ranges of | | |
| | | 0.5-2, 0.5-1-1-2 | | |
| alkaline | 80-300 | ranges of 50- | no worsening | no worsening, 1 |
| phospatase | | 600 | | to about 90% |
| (ALP) | | | | reduction, |
| (80-260 IU/L) | | | | 300 IU/L or less, |
| | | | | 250 IU/L or less |
| Total bilirubin | | | no worsening | no worsening, 1 |
| (0.2-1.2 | | | | to about 90% |
| mg/dL) | | | | reduction |
| Gamma- | | | no worsening | no worsening, 1 |
| Glutamyl | | | | to about 90% |
| Transferase | | | | reduction, |
| (GGT or γGTP) | | | | 100 U/L or less, |
| (males: 5-60 | | | | 70 U/L or less |
| U/L) | | | | |
| Albumin (3.8- | | | no worsening | no worsening, 1 |
| 5.2 g/dl) | | | | to about 90% |
| | | | | increase, ranges |
| | | | | of 3-6 g/dl, 3.5- |
| | | | | 5.5 g/dl |
| HDL-C (high | less than 55 | less than | no worsening, at | no change, 1- |
| density | | 60 mg/dl, 55, 50, | least 1% | 90% increase, |
| lipoprotein | | 45, 40, 35, 30, | increase | 40 mg/dl or |
| cholesterol) | | 25, or 25 mg/dl; | | more |
| (35-60- mg/dl) | | ranges of 25-55, | | |
| | | 30-40 mg/dl, 40- | | |
| | | 50 mg/dl, 50- | | |
| | | 60 mg/dl, at | | |
| | | least 60 | | |
| LDL-C (low | 100-200 | at least 70 | no worsening | no change, 1- |
| density | | mg/dl, 100, | | 90% reduction |
| lipoprotein | | 120, 130 140 | | less than |
| cholesterol) | | 150, 170, 190, | | 160 mg/dl, 140, |
| (50-130 | | or 200 or a | | 130, 120, 100, |
| mg/dl) | | range of 70-300, | | 70 mg/dl |
| | | 70-250, 70-200, | | |
| | | 100-250, 100- | | |
| | | 200, 130-200, | | |
| | | 140-180, 100- | | |
| | | 130, 130-160, | | |
| | | 160-190 | | |
| Triglycerides | 100-1000 | at least 80 | at least 1% | 1 to about 90% |
| (TG) (fed or | | mg/dl, 100, 150, | lower | reduction, 500 |
| fasting, 50- | | 180, 200, 300, | | mg/dl or less, |
| 150 mg/dl) | | 500, 700, 1000, | | 300, 200, 150, |
| | | 1200, or 1500, | | 100 mg/dl or |
| | | or less than 150, | | less |
| | | or a range of | | |
| | | 100-2500, 100- | | |
| | | 1500, 100-1000, | | |
| | | 150-500, 200- | | |
| | | 500, 150-300, | | |
| | | 150-200, 200- | | |
| | | 500 | | |
| Total | 170-300 | a range of 130- | no worsening | no change, 1- |
| Cholesterol | | 300 mg/dl, 200- | | 90% reduction |
| (TC) (100-200 | | 220, 220-240, | | |
| mg/dl) | | 240-260, or at | | |
| | | least 260, or less | | |
| | | than 200 mg/dl | | |
| TG and HDL-C | High TG and | TG: at least 150, | no worsening | |
| | low HDL-C | 200, 500 mg/dl | | |
| | (ex. TG ≧ | HDL-C; less than | | |
| | 150 mg/dl and | 40, 50 mg/dl | | |
| | HDL ≦ 40 mg/dl | | | |
| TG/HDL-C | at least 3.75 | at least 2, 2.5, 3, | at least 1% | no worsening, at |
| ratio | | 3.75, 4, 5, 10, or | lower | least 1% lower, or |
| | | ranges of | | 1-90% reduction |
| | | 2-3.75, 3.75-10 | | |
| Non-HDL-C | at least 130 | at least | no worsening | no worsening, |
| (mg/dl) | | 100 mg/dl, 130, | | or at least 1% |
| | | 150, 160, 170, | | lower, or less |
| | | 190, a range of | | than 130 mg/dl, |
| | | 100 to 250 | | 150, 160, 170, |
| | | | | 190 |
| Free fatty acid | at least 400 | less than 400, at | at least 1% | no change, or at |
| (μ Eq/l) | | least 400, 600, | lower | least 1 to 90% |
| (140-850) | | 800, 1000 | | reduction |
| Eicosapentaenoic | less than 0.5/low | less than 1, | at least 5% | 5 to about 200% |
| Acid/Arachidonic | compared to | 0.75, 0.5, 0.1, | increase | increase, about |
| Acid | average level | ranges of 0.01-2 | | 2-200-fold |
| (EPA/AA) | or normal | | | increase |
| (ex. (mol/%)/ | subjects | | | |
| (mol/%) | | | | |
| Arachidonic | High | | at least 1% | no change, 1 to |
| Acid (AA) | compared to | | lower | about 90% |
| (ex. mol/%) | average level | | | reduction |
| | of normal | | | |
| | subjects | | | |
| Eicosapentaenoic | low | | at least 5% | 5 to about 200% |
| Acid (EPA) | compared to | | increase | increase, about |
| (ex. mol/%) | average level | | | 2-500-fold |
| | of normal | | | increase |
| | subjects | | | |
| Docosapentaenoic | low | | at least 1% | 1 to about 95% |
| Acid | compared to | | increase | increase |
| (DPA) | average level | | | |
| (ex. mol/%) | of normal | | | |
| | subjects | | | |
| Docosahexaenoic | low | | | |
| Acid (DHA) | compared to | | | |
| (ex. mol/%) | average level | | | |
| | of normal | | | |
| | subjects | | | |
| DPA/AA ratio | low | | | |
| | compared to | | | |
| | average level | | | |
| | of normal | | | |
| | subjects | | | |
| DPA/AA ratio | low | | | |
| | compared to | | | |
| | average level | | | |
| | of normal | | | |
| | subjects | | | |
| DHA/DPA | low | | | |
| ratio | compared to | | | |
| | average level | | | |
| | of normal | | | |
| | subjects | | | |
| Monounsaturated | High | | at least 1% | no change, at |
| fatty acid | compared to | | lower | least 1% lower |
| (MUFA) | average level | | | |
| (ex. mol/%) | of normal | | | |
| | subjects | | | |
| Palmitoleic | High | | at least 1% | no change, at |
| acid (16:1 n7) | compared to | | lower | least 1% lower |
| (ex. mol/%) | average level | | | |
| | of normal | | | |
| | subjects | | | |
| Oleic acid | High | | at ieast 1% | no change, a |
| (18:1 n9) (ex. | compared to | | lower | least 1% lower |
| mol/%) | average level | | | |
| | of normal | | | |
| | subjects | | | |
| Oleic acid | High | | at least 1% | no change, at |
| (18:1 n9)/ | compared to | | lower | least 1% lower |
| stearic acid | average level | | | |
| (18:0) ratio | of normal | | | |
| | subjects | | | |
| Palmitoleic | High | | at least 1% | no change, at |
| acid (16:1)/ | compared to | | lower | least 1% lower |
| Palmitic acid | average level | | | |
| (16:0) ratio | of normal | | | |
| | subjects | | | |
| Stearic acid | High | | no change, or at | no change, or at |
| (18:0)/ | compared to | | least 1% lower | least 1% lower |
| Palmitic acid | average level | | | |
| (16:0) ratio | or normal | | | |
| | subjects | | | |
| γ-linoleic | High | | no change, or at | no change, or at |
| acid(18:3 n6)/ | compared to | | least 1% lower | least 1% lower |
| Linoleic acid | average level | | | |
| (18:2 n6) ratio | subjects | | | |
| AA/Homo-γ- | low | | no change, or at | no change, or at |
| linolenic acid | compared to | | least 1% | least 1% |
| (20:3 n6) ratio | average level | | Increase | increase |
| | of normal | | | |
| | subjects | | | |
| Acrenic acid | High | | no change, or at | no change, or at |
| (22:4 n6)/ | compared to | | least 1% lower | least 1% lower |
| AA ratio | average level | | | |
| | of normal | | | |
| | subjects | | | |
| Ferritin | | at least 100, | at least 1% | at least 1 to |
| (ng/mL) | | 120, 150, 200, | lower | about 95% |
| | | 250, 300, 350, | | lower |
| | | 400, or 500 | | |
| Thioredoxin | | at least 15, 20, | at least 1% | at least 1 to |
| (ng/mL) | | 25, 30, 35, 40, | lower | about 95% |
| | | 45, or 50 | | lower |
| TNFα (pg/mL) | at least 1.5 | at least 1, 1.5, | at least 1% | at least 1 to |
| (1.79 or less) | | 1.6, 1.7, 1.79, | lower | about 95% |
| | | 1.8, 1.9, 2.0, 2.2, | | lower |
| | | 2.5, 3, 3.5, 4, 5, | | |
| | | 6, 7 or 10 | | |
| sTNF-R1 | | at least 400, | at least 1% | at least 1 to |
| (pg/mL) | | 500, 600, 700, | lower | about 95% |
| | | 800, 900, 1000, | | lower |
| | | 1100, 1200, | | |
| | | 1500, or 2000 | | |
| sTNF-R2 | | at least 500, | at least 1% | at least 1 to |
| (pg/mL) | | 700, 1000, 1200, | lower | about 95% |
| | | 1500, 1700, | | lower |
| | | 2000, 2200, | | |
| | | 2500, 2700, or | | |
| | | 3000 | | |
| High | 0.2 | 0.1 or more, 0.2, | at least 1% | at least 5 to |
| Sensitivity C- | | 0.3, 0.4, 0.5 or | lower | about 95% |
| reactive | | more, ranges of | | lower |
| protien (Hs- | | 0.1-1, 0.1-0.8, | | |
| CRP, mg/dl) | | 0.1-0.5, 0.2-0.5 | | |
| Connective | | | at least 1% | at least 5 to |
| Tissue Growth | | | lower | about 95% |
| Factor (CTGF) | | | | lower |
| Serum Soluble | | 5 pg/ml or | at least 1% | at least 5 to |
| CD40 (sCD40, | | more, 10, 20, | lower | about 95% |
| pg/ml) | | 30, 50, 70, 100, | | lower |
| | | 120, 150, 170, | | |
| | | 200, 220, 250, | | |
| | | 300, 350, 400, | | |
| | | 450, 500 or | | |
| | | more | | |
| Insulin | 1.5 or more | 1.6 or less/1.5 | no worsening | no change, at |
| resistance | | or more, 1.6, 2, | | least 1 to about |
| Index (HOMA- | | 2.5, 3, 3.5, 4 | | 50% lower |
| IR) (1.6 or | | | | |
| less) | | | | |
| Glycated | 5.7 or more | a range of 4.3- | no worsening | no change, at |
| hemoglobin | | 5.8, 5.7-6.4, 5.8- | | least 1 to about |
| (HbA1c) (4.3- | | 6.5, 6.5-7.0, 7.0- | | 50% lower |
| 5.8%) | | 8.0/5.7 or | | |
| | | more, 5.8, 6, | | |
| | | 6.5, 7, 7.5, 8, or | | |
| | | 8.5 | | |
| Fasting | 100 or more | less than 100/ | no worsening | no change, or at |
| plasma | | 100 or more, | | least 1 to about |
| glucose (FPG) | | 110, 120, 126, | | 50% lower |
| (mg/dl) | | 130, 150, 200, | | |
| (less than 100) | | 250, 300/ | | |
| | | ranges of 100- | | |
| | | 110, 100-126 | | |
| Postprandial | 140 or more | less than 140, | no worsening | no change, or at |
| plasma | | 160, 200/ | | least 1 to about |
| glucose (after | | 140 or more, | | 50% lower |
| a meal) | | 170, 180, 200, | | |
| | | 250, 300, 350 | | |
| | | 400/ranges of | | |
| | | 140-200, 140- | | |
| | | 170, 170-200 | | |
| two-hour | 140-200 | less than 140, | no worsening | no change, or at |
| glucose levels | | 160, 200/140 or | | least 1 to about |
| on the 75-g | | more, 170, 180, | | 50% lower |
| oral glucose | | 200, 250, 300, | | |
| tolerance test | | 350, 400/ | | |
| (mg/dl) | | ranges of 140- | | |
| (OGTT) | | 200, 140-170, | | |
| | | 170-200 | | |
| Leptin (ng/ml) | | 5 ng/ml or | at least 1% | at least 1 to |
| | | more, 10, 12, | lower | about 95% |
| | | 15, 17, 20, 22, | | reduction |
| | | 25, 30, 35, 40 | | |
| | | or more | | |
| Serum | | 5 μg/mL or less, | at least 1% | no change, at |
| adiponectin | | 4.5, 4, 3.5, or 3 | increase | least 1 to about |
| (μg/mL) | | μg/mL or less | | 95% increase |
| complement | | | at least 15% | at least 1 to |
| factor D | | | lower | about 95% |
| | | | | reduction |
| CK18 | | | at least 1% | at least 1 to |
| fragment | | | lower | about 95% |
| | | | | reduction |
| serum High | | | at least 1% | at least 1 to |
| mobility group | | | lower | about 95% |
| box 1 protein | | | | reduction |
| (HMGB1) | | | | |
| Fas | | | at least 1% | at least 1 to |
| | | | lower | about 95% |
| | | | | reduction |
| Hyaluronic | | 25 ng/mL or | at least 1% | at least 1 to |
| acid | | more, 50, 70, | lower | about 95% |
| (50 ng/mL or | | 100, 120, 150, | | reduction |
| less) | | 200, 250, or 300 | | |
| | | or more; 200 mL | | |
| | | or less, 100, 70, | | |
| | | or 50 or less | | |
| Type IV | | 5 ng/mL or | at least 1% | at least 1 to |
| collagen (7s | | more, 6, 7, 8, | lower | about 95% |
| domain) | | 10, 12, 15, or 20 | | reduction |
| (6 ng/mL or | | or more; | | |
| less) | | 25 ng/mL or | | |
| | | less, 20, 15, 10, | | |
| | | or 6 or less | | |
| procollagen III | | 0.2 U/ml or | at least 1% | at least 1 to |
| peptide 0.3- | | more, 0.3, 0.5, | lower | about 95% |
| 0.8 U/ml | | 0.7, 1, 1.2, 1.5, | | reduction |
| | | 2, 2.5, 3, 3.5, or | | |
| | | 4 or more; 10 or | | |
| | | less, 8, 5, 3, 1, or | | |
| | | 0.8 or less | | |
| PAI-1 (ng/mL) | 50 or more | | | |
| 50 or less | | | | |

Items other than serum

| | | | | |
|---|---|---|---|---|
| platelet count | 150000-300000 | 400000/μl or | no change | no change, at |
| 150000- | | less, 300000, | | least 1% |
| 400000/μl | | 200000/a range | | increase |
| | | of 150000-300000 | | |
| BMI | 18.5-40 | 18.5 or more, | no change | no change, at |
| | | 20, 25, 30, 35, | | least 1% |
| | | 40, or 50 or | | reduction |
| | | more;/50 or | | |
| | | less, 40, 30, 25, | | |
| | | 20 or 18.5 or | | |
| | | less; or range of | | |
| | | 18.5-25, 25-30, | | |
| | | 30-35. 35-40 | | |

### Example

### Treatment of NASH

To evidence the usefulness of the present invention for the treatment of NASH, patients are evaluated for inclusion in the treatment regimen, treated for NASH, and evaluated for effectiveness of the treatment as follows:

Patients are histologically diagnosed with NASH within six months of the initiation of treatment and are willing to submit to a further liver biopsy at the end of the treatment regimen to evaluate effectiveness of the treatment.

1. Inclusion Criteria:

Patients are definitively diagnosed with NASH (via liver biopsy) and exhibit a NAS score of greater than or equal to 4 by a pathologist.
Patients can be of either gender but are greater than 18 years of age.
Patients with diabetes, impaired glucose tolerance or metabolic syndrome that have been on stable dosage of anti-diabetic agents for at least six months prior to the liver biopsy are suitable for treatment.

2. Exclusion Criteria:

Patients may be excluded for treatment based upon an inability or unwillingness to have a liver biopsy for confirming the diagnosis of NASH, having a diagnosis of cirrhosis by pathologist, exhibiting previous bariatric surgery or biliary diversion (i.e. gastric bypass), esophageal banding or gastric banding; serum ALT values of greater than 330 UL, drug use associated with steatohepatitis within 6 months prior to initiation of treatment, such as with corticosteroids, high dose estrogens, methodtrexate, amiodarone, anti-HIV drugs, tamoxifen, or diltiazem; alcohol consumption of greater than 30 g/day, concurrently or for more than three consecutive months within five years prior treatment; a blood alcohol level greater than 0.02% at the time of baseline evaluation; evidence of active substance abuse; including prescription or recreational drugs, the presence of other liver diseases such as acute or chronic hepatitis C, acute or chronic active hepatitis B, Wilson's, autoimmune, alpha-1-antitrypsin and hemochromatosis or HIV infection; renal insufficiency; symptomatic coronary; peripheral or neurovascular disease; symptomatic heart failure or advanced respiratory disease requiring oxygen therapy; a history of cerebral or retinal hemorrhage or other bleeding diathesis.

3. Key Criteria for Measuring Baseline and Post Treatment Values:

Patients to be treated are evaluated for one or more of the following criteria.

a) Primary Long-Term Efficacy Outcome Measure
Histology at treatment month 12.5 to evaluate the NAS score, as a comparison to the baseline score measured pre-treatment. (NAS)

b) Primary Short-Term Efficacy Outcome Measure
Change from baseline in ALT levels at Month 3 and Month 6 of treatment.

c) Secondary Efficacy Outcome Measures
Overall NAS score
Feature scores including fibrosis, ballooning degeneration, inflammation and steatosis
Liver function tests (AST, alkaline phosphataise, bilirubin, GGT, Albumin)
Cholesterol (including HDL and LDL)
Triglycerides
Fatty acid assay
Ferritin
Thioredoxin
Pro-inflammatory cytokines (TNF-α, sTNF-R1, sTNF-R2, Hs-CRP, CTGF, sCD40)
Insulin sensitivity (HOMA-IR)
HbAlc
Glucose
Leptin, Serum adiponectin and complement factor D
CK18 fragment and Serum HMGB1
Fas
Hyaluronic acid
Type IV collagen (7S domain)
Procollagen III peptide

d) Safety Outcome Measures
Adverse Events
Hematology/biochemistry/urinalysis
ECG (including QT/QTc measurement)

e) Pharmacokinetic Outcome Measures
EPA, DPA and DHA
Day 1
   On Day 1, samples for plasma concentration are obtained at predose and 0.5, 1, 2, 4, 5 and 6 hours after Dose #1 and Dose #3; after Dose #2, samples are obtained at 2, 4, 5 and 6 hours post-dose. After Dose #3, samples are also obtained at 8 and 12 hours post-dose (20 and 24 hours after Dose #1 [prior to the morning dose on Day 2])
   Cₘₐₓ (Dose #1 and Dose #2s) and Cₘₐₓ, Tₘₐₓ, T_{1/2}, AUG₀₋ₜ after third Dose are derived from plasma concentrations
Days 29, 85, 169 and 365 (Visits 3, 5, 7 and 9)
   A single sample is obtained prior to the morning dose (trough) on Visits 3, 5, 7 and 9.
   Css is determined from plasma concentrations

4. Concomitant and Medications:

Particular medications can be prohibited or permitted during treatment according to the invention for NASH.

The following medications can be prohibited during treatment:
Omega-3-acid ethyl esters and omega-3-PUFA containing supplements>200 mg per day
Vitamin E>60 IU per day
Thiazolidinediones (e.g. pioglitazone, rosiglitazone)

The following medications may be used during the treatment according to the specified restrictions:
Subjects may continue prescription or over-the-counter medications or herbal remedies such as HMG-CoA reductase inhibitors (stains), fibrates, probucol, ezetimibe, ursodiol (UDCA), taurine, betaine, N-acetylcysteine, s-adenosylmethionine (SAM-e), milk thistle, anti-TNF therapies, or probiotics
Subjects may continue the following anti-diabetic medications: biguanides (metformin), insulin, sulfonylureas, alpha-glucosidase inhibitors (acarbose), dipeptidyl-peptidase 4 inhibitors (sitagliptin, saxagliptin), and phenylalanine derivatives (nateglinide, repaglinide)
Subjects may continue receiving anti-platelet therapy and anti-thrmobotic agents (e.g. warfarin, ASA, and clopidogrel) after study commencement should be monitored closely during the study for bleeding problems.

5. Treatment

Patients are treated with EPA-E comprised of two daily treatments, but the total daily dose of EPA-E being 1800 mg or 2700 mg per day, divided into dosage amounts of 600 mg TID or 900 mg TID, respectively.

Treatment with EPA-E is continued for 12 months.

Patients are periodically evaluated for the selected criteria, such as at month 1, month 3, month 6 and month 12 of treatment.

After 12 months of treatment, patients are evaluated for the criteria noted above, including liver biopsy, NAS score, steatosis, lobular inflammation, ballooning and fibrosis stage, and one or more of the other criteria listed above in Table 1.

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

We claim:

1. A method for treating NASH in a subject in need thereof, comprising:
(a) identifying a subject having NASH;
(b) determining the baseline level in said subject of at least one criteria selected from the group consisting of NAS score, steatosis score, lobular inflammation score, ballooning score and fibrosis stage; and
(c) administering to said subject an effective amount of ethyl eicosapentanoate (EPA-E).

2. The method according to claim 1, wherein said subject has a NAS score of ≧4.

3. The method according to claim 1 or 2, wherein said subject is characterized by at least one criteria selected from the group consisting of a baseline ALT value of about 10 to about 300 U/L; a baseline AST value of about 10 to about 250 U/L; a baseline steatosis grade of about 2 to 3; and a baseline lobular inflammation grade of about 2 to 3.

4. The method according to claim 3, wherein after said administration of said EPA-E for about one year, said subject exhibits at least one improvement selected from the group consisting of a reduced ALT value as compared to said baseline ALT value; a reduced AST value as compared to said baseline AST value; a reduced steatosis grade as compared to said baseline steatosis grade; and a reduced lobular inflammation grade as compared to said baseline lobular inflammation grade.

5. The method according to claim 4, wherein said ethyl eicosapentanoate is administered to said subject in an amount of between about 1800 and about 2700 mg per day.

6. The method according to claim 1, wherein said subject is further characterized by having at least one condition selected from the group consisting of high TG and low HDL-C, diabetes, impaired glucose tolerance and metabolic syndrome.

7. The method according to claim 4, wherein said reduced ALT value is at least 5% lower than said baseline ALT value and/or said reduced AST value is at least 5% lower than said baseline AST value.

8. The method according to claim 1, further comprising determining in said subject prior to treatment a baseline level in serum of at least one member selected from the group consisting of ALT in a range of 10 to 300 U/L, AST in a range of 10 to 250 U/L, HDL-C in a range of 25 to 55 mg/dl, LDL-C in a range of 100 to 200 mg/dl, triglycerides in a range of 100 to 1000 mg/dl, TC in a range of 170 to 300 mg/dl, High TG and low HDL-C, TG/HDL-C ratio in a range of 3.75 to 10, non-HDL-C in a range of 100 to 250 mg/dl, Free fatty acid in a range of 400 to 1000 μ Eq/L, HOMA-IR in a range of 1.5 to 5, HbA1c in a range of 5.7 to 10%, Fasting plasma glucose in a range of 100 to 200 mg/dl.

9. The method according to claim 8, wherein after administration of ethyl eicosapentanoate for at least 3 months, said subject exhibits the following changes in said at least one marker as compared to the baseline level of at least 1% reduction for ALT, AST, TG, TG/HDL ratio, Fee fatty acid, AA, MUFA, Palmitoleic acid, Oleic acid, Oleic acid/Stearic acid ratio, Palmitoleic acid/Palmitic acid ratio, Adrenic acid/AA ratio, Ferritin, Thioredoxin, TNF α, sTNF-R1, sTNF-R2, Hs-CRP, CRGF, sCD40, Leptin, complement factor D, CK18 fragment, serum HMGB1, Fas, Hyaluronic acid, Type IV collagen (7s domain), procollagen III peptide or PAI-1; at least 5% increase for EPA or EPA/AA ratio; at least 1% increase for DPA, AA/Homo-γ-linoleic acid ratio or Serum adiponectin; no worsening of ALP, bilirubin, GGT, Albumin, HDL-C, LDL-C, TC, non-HDL-C, HOMA-IR, HbA1c, Glucose, Fasting plasma glucose, postprandial plasma glucose, OGTT, platelet count or BMI.

10. The method according to claim 1, further comprising: (d) improving the NAS score in said subject (i) to a composite score of ≦3 and no worsening of said fibrosis stage score, or (ii) by ≧2 across at least two of the NAS components and no worsening of said fibrosis stage score.

11. A method for treating NASH in a subject in need thereof, comprising:
(a) identifying a subject having NASH;
(b) determining the baseline level in said subject of at least one criteria selected from the group consisting of NAS score, steatosis score, lobular inflammation score, ballooning score and fibrosis stage;
(c) administering to said subject an effective amount of ethyl eicosapentanoate (EPA-E); and
(d) improving the NAS score in said subject (i) to a composite score of ≦3 and no worsening of said fibrosis stage score, or (ii) by ≧2 across at least two of the NAS components and no worsening of said fibrosis stage score.

12. The method according to claim 11, wherein said subject has a baseline NAS score of ≧4.

13. The method according to claim 12, wherein after said administration of said EPA-E once daily for about one year, said subject exhibits at least one improvement selected from the group consisting of a reduced ALT value as compared to said baseline ALT value; a reduced AST value as compared to said baseline AST value; and a reduced lobular inflammation grade as compared to said baseline lobular inflammation grade.

14. The method according to claim 13, wherein said reduced ALT value is at least 10% lower than said baseline ALT value and/or said reduced AST value is at least 10% lower than said baseline AST value.

15. The method according to claim 12, wherein after administration of ethyl eicosapentanoate for at least 12 months, said subject exhibits at least 10% reduction as compared to the baseline level of at least one marker selected from the group consisting of ALT, AST, TG, Ferritin, Thioredoxin, TNF-α, hyaluronic acid and Type IV collagen (7S domain); at least 5% reduction for HDL, LDL, EPA/AA, AA, DPA, STNF-R1, STNF-R2, HSCRP, CTGF, SCD40, Leptin, Seum adiponectin, complement factor D, CK18 fragment, serum HMGB1, Fas or procollegen III peptide and no worsening of HOMA-IR, HbA1c, glucose, platelet count or BMI.

16. A method for treating NASH in a subject in need thereof, comprising:
(a) identifying a subject having NASH characterized by baseline levels in said subject of ALT of between 5 to 300 and at least one criteria selected from the group consisting of NAS score of ≧4, steatosis score of 1, lobular inflammation score of ≧1 and either (i) fibrosis stage of at least 1a or ballooning; and
(c) administering to said subject an effective amount of ethyl eicosapentanoate (EPA-E); and
(d) improving the NAS score in said subject (i) to a composite score of ≧3 and no worsening of said fibrosis stage score, and (ii) by ≧2 across at least two of the NAS components and no worsening of said fibrosis stage score.

17. The method according to claim 16, wherein said ethyl eicosapentanoate is administered to said subject in an amount of between about 1800 and about 2700 mg per day.

18. The method according to claim 17, wherein after administration of ethyl eicosapentanoate for at least 12 months, said subject exhibits at least 10% reduction as compared to the baseline level of at least one member of the group consisting of ALT, AST, TG, Ferritin, Thioredoxin, TNF-α, hyaluronic acid or Type IV collagen (7S domain),
at least 5% reduction for HDL, LDL, EPA/AA, AA, DPA, STNF-R1, STNF-R2, HSCRP, CTGF, SCD40, Leptin, Seum adiponectin, complement factor D, CK18 fragment, serum HMGB1, Fas or procollegen III peptide and no worsening of HOMA-IR, HbA1c, glucose, platelet count or BMI.

19. The method according to claim 18, wherein said EPA-E is administered twice daily in dosage amounts of 600 mg or 900 mg.

20. A method for treating NASH in a subject in need thereof, comprising:
(a) administering to a subject an effective amount of ethyl eicosapentanoate (EPA-E), wherein said subject has NASH and is characterized by baseline levels in said subject of ALT of between 5 to 300 and at least one criteria selected from the group consisting of NAS score of 4, steatosis score of ≧1, lobular inflammation score of ≧1 and either (i) fibrosis stage of at least 1a or (ii) ballooning; and
(b) improving the NAS score in said subject (i) to a composite score of ≦3 and (ii) by ≧2 across at least two of the NAS components, and no worsening of said fibrosis stage score.

21. The method according to claim 20, wherein after administration of ethyl eicosapentanoate for at least 12 months, said subject exhibits at least 10% reduction as compared to the baseline level for at least one member selected from the group consisting of ALT, AST, TG, Ferritin, Thioredoxin, TNF-α, hyaluronic acid or Type IV collagen (7S domain); at least 5% reduction for HDL, LDL, EPA/AA, AA, DPA, STNF-R1, STNF-R2, HSCRP, CTGF, SCD40, Leptin, Seum adiponectin, complement factor D, CK18 fragment, serum HMGB1, Fas or procollegen III peptide and no worsening of HOMA-IR, HbA1c, glucose, platelet count or BMI.

22. A method for reducing steatosis, liver lobular inflammation and/or liver fibrosis in a subject in need thereof, comprising:
(a) administering to a subject an effective amount of ethyl eicosapentanoate (EPA-E);
(b) improving the steatosis and lobular inflammation condition of said subject, and no worsening of said fibrosis stage score; and
(c) said subject exhibits the following changes in said at least one marker as compared to a baseline pretreatment level of at least 1% reduction for ALT, AST, TG, TG/HDL ratio, Free fatty acid, AA, MUFA, Palmitoleic acid, Oleic acid, Oleic acid/Stearic acid ratio, Palmitoleic acid/Palmitic acid ratio, Stearic acid/Palmitic acid ratio, y-linoleic acid/Linoleic acid ratio, Adrenic acid/AA ratio, Ferritin, Thioredoxin, TNF α, sTNF-R1, sTNF-R2, Hs-CRP, CTGF, sCD40, Leptin, complement factor D, CK18 fragment, serum HMGB1, Fas, Hyaluronic acid, Type IV collagen (7s domain), procollagen III peptide or PAI-1; at least 5% increase for EPA or EPA/AA ratio; at least 1% increase for DPA, AA/Homo-γ-linoleic acid ratio or Serum adiponectin; no worsening of ALP, bilirubin, GGT, Albumin, HDL-C, LDL-C, TC, non-HDL-C, HOMA-IR, HbA1c, Glucose, Fasting plasma glucose, postprandial plasma glucose, OGTT, platelet count or BMI.

23. The method according to claim 22, wherein said ethyl eicosapentanoate is administered to said subject in an amount of about 1800 or about 2700 mg per day.

24. A method for treating NASH in a subject in need thereof, comprising: administering to a subject an effective amount of EPA-E, wherein the subject is possible or definite NASH, and is characterized by the baseline pretreatment level in the subject of at least one criteria selected from the group consisting of ALT in a range of 10 to 300 U/L, AST in a range of 10 to 250 U/L, HDL/C in a range of 25 to 55 mg/dl, LDL-C in a range of 100 to 200 mg/dl, triglycerides in a range of 100 to 1000 mg/dl, TC in a range of 170 to 300 mg/dl, High TG and low HDL-C, TG/HDL-C ratio in a range of 3.75 to 10, non-HDL-C in a range of 100 to 250 mg/dl, Free fatty acid in a range of 400 to 1000 μ Eq/L, HOMA-IR in a range of 1.5 to 5, HbA1c in a range of 5.7 to 10%, Fasting plasma glucose in a range of 100 to 200 mg/dl, impaired glucose tolerance and metabolic syndrome.

25. A method for treating NASH in a subject suspected of having NASH, comprising: administering to a subject an effective amount of EPA-E, wherein the subject is possible or definite NASH, and is characterized by the baseline pretreatment level in the subject of at least one criteria selected from the group consisting of low level of EPA, DPA, DHA, EPA/AA, DHA/AA. DHA/DPA, AA/Homo-γ-linoleic acid: and high level of AA, MUFA, Palmitoleic acid, Oleic acid, Oleic acid/Stearic acid, Palmitoleic acid/Palmitic acid, γ-linoleic acid/Linoleic acid, Adrenic acid/AA compared to each average level in subjects with NASH.
